(19) United States
(12) Patent Application Publication
(10) Pub. No.: US 2015/0082467 A1
(43) Pub. Date: Mar. 19, 2015

(54) PHARMACEUTICAL COMPOSITION FOR VIRAL TREATMENT, AND METHOD FOR SCREENING ANTIVIRAL AGENT

(71) Applicant: Industry-Academic Cooperation Foundation, Yonsei University, Seoul (KR)

(72) Inventors: Young-Joon Kim, Sinsa-dong Gangnam-gu Seoul (KR); Myeong Sup Lee, Bangbae-dong Seocho-gu Seoul (KR); Byungil Kim, Guro 5-dong Guro-gu Seoul (KR); Goo Taeg Oh, Seodaemun-gu (KR)

(21) Appl. No.: 14/376,987

(22) PCT Filed: Feb. 13, 2013
(86) PCT No.: PCT/KR2013/001111

§ 371 Date: Aug. 6, 2014

(30) Foreign Application Priority Data
Feb. 13, 2012 (KR) 10-2012-0014516

(51) Int. Cl. C12N 15/113; A01K 67/027; C12Q 1/68; G01N 33/573; G01N 33/50

(52) U.S. Cl. CPC C12N 15/1137; G01N 33/573; G01N 33/5008; C12Q 1/6883; A01K 67/0276; A01K 2267/0337; A01K 2227/105; G01N 2333/9125; C12Q 2600/158; C12Q 2600/136
USPC 800/9; 536/24.5; 536/24.33; 435/7.4; 435/6.13; 435/5; 435/6.12; 435/6.11

(57) ABSTRACT

The present invention relates to; a pharmaceutical compostion capable of enhancing immunity against viruses by specifically decreasing the expression of the OASL1 protein; and a method for screening for a material capable of being used as an antiviral agent by comparing the amount of expression of the OASL1 protein.

### CROSS-REFERENCE TO RELATED APPLICATIONS

This is a National Phase application claiming priority to PCT/KR2013/001111 filed Feb. 13, 2013, which claims priority to KR 10-2012-0014516 filed Feb. 13, 2012, which are hereby incorporated by reference in their entirety.

### FIELD OF THE INVENTION

The present invention relates to a pharmaceutical composition for viral treatment and a method for screening an antiviral agent.

### BACKGROUND OF THE INVENTION

After infection with viruses, the pattern-recognition receptors (PRRs), displayed in the intracellular space and on the plasma membrane of immune cells like macrophages and dendritic cells, recognize conserved pathogen-associated molecular patterns (PAMPs) of the host, thus recognizing the pathogen, and initiate the inflammatory response, which is essential in an early stage of eliminating the pathogen.

Toll-like receptors (TLRs) are typical transmembrane PRRs that recognize viruses. TLR3 recognizes double-stranded RNA (dsRNA) and polyinosinic-polycytidylic acid (poly (I:C)), which is a synthetic analog of dsRNA; TLR7 recognizes single-stranded RNA (ssRNA) and imidazoquinoline resiquimod (R848); and TLR9 recognizes CpG DNA. Cytoplasmic PRRs have i) RIG-I-like receptors (RLRs), such as retinoic acid-inducible gene I (RIG-I) and melanoma differentiation associated gene 5 (MDA5), which recognize dsRNA and poly (I:C); ii) DNA-dependent activator of IRFs (DAI, or ZBP1), which recognizes DNA that is rich in AT base pairs, for example, poly(dA)·poly(dT)(dAdT); and iii) interferon (IFN)-inducible gene 16 (IFI16), which recognizes DNA that is not rich in AT base pairs.

Such PRRs initiate various signal transductions and, as a result, produce two essential mediators of the inflammatory response. The first of these mediators are inflammatory cytokines, such as tumor necrosis factor-alpha (TNFα), and they initiate and amplify the inflammatory response. The second of these mediators are type I interferons, such as IFNαs/β, and they suppress virus replication in the host. Here, the transcription factor (TF) nuclear factor-kappa B (NF-κB) plays a key role in the expression of inflammatory cytokines and may facilitate the expression of IFNβ.

Interferon regulatory factors 3 and 7 (IRF3 and IRF7) are the main transcription factors that can induce the expression of type I interferons in inflammatory cells. IRF3 is constitutively expressed and, after virus infection, is activated and undergoes translocation into the nucleus, where it acts as the key transcription factor for the early expression of IFNβ and IFNα4. IRF7 is weakly expressed in most cells; after virus infection, however, the expression is strongly induced by type I interferon-mediated positive feedback loop signaling and IRF7 is activated similarly as IRF3. Afterwards, IRF7 undergoes translocation into the nucleus, where it acts as the key transcription factor for the expression of IFNαs, and also, by forming a heterodimer with IRF3, participates in the expression of IFNβ in a crucial way. Therefore, IRF7 is known to play the most critical role in the overproduction of type I interferons during virus infection.

In most cells, the type I interferon induces an antiviral state through a large number of IFN-stimulated genes (ISGs) and mediates diverse antiviral pathways. RNase L is activated by the 2′-5′-oligoadenylate (2-5A), which is produced by activated 2′-5′-oligoadenylate synthetase (OAS). The activated RNase L is well known to activate an antiviral mechanism by degrading cellular and viral RNA. The OAS family comprises a dozen proteins in mice. However, as many OAS family proteins do not produce 2-5A, other functions of nonenzymatic OAS proteins are being conjectured. OAS 1d, a nonenzymatic OAS protein, is involved in the development of germ cells, and OAS1b, another nonenzymatic protein, confers resistance to certain viruses, such as West Nile virus.

OASL1, which is yet another nonenzymatic OAS protein, remains largely unknown. The OASL1 protein has the OAS domain and dsRNA-binding site like other OAS proteins, but additionally has two ubiquitin (Ub)-like domains.

It has also been shown that if the amount of expression of type I interferons increases in vivo, then the antibody production capacity in vivo is substantially enhanced (Le Bon, Agnes, et al. “Type I Interferons Potently Enhance Humoral Immunity and Can Promote Isotype Switching by Stimulating Dendritic Cells In Vivo.” Immunity, Vol. 14, 461-470 (April, 2001); Le Bon, Agnes, et al. “Cutting Edge: Enhancement of Antibody Responses Through Direct Stimulation of B and T Cells by Type I IFN.” J. Immunol., 176, 2074-2078 (2006)).

### DETAILED DESCRIPTION OF THE INVENTION

### Technical Problems

The present invention aims to elucidate the properties associated with antiviral mechanisms of OASL1 and, in so doing, to provide an antiviral agent and a method for screening an antiviral agent.

### Technical Solutions

An embodiment of the present invention provides an antiviral pharmaceutical composition comprising, as active ingredients, antisense or siRNA oligonucleotide that has a sequence complementary to a nucleotide sequence of the Oasl1 gene. In the embodiment, the nucleotide sequence of the Oasl1 gene can be any one of SEQ ID NOs: 1 to 7.

Another embodiment of the present invention provides a method for screening an antiviral agent, comprising the steps of: (a) measuring the amount or activity of OASL1 protein in cells; (b) injecting into cells a sample to be assayed; (c) measuring the amount or activity of OASL1 protein in cells of step (b); and (d) determining the sample to be assayed as an antiviral agent if the amount or activity of OASL1 protein in step (c) is less than the amount or activity of OASL1 protein in step (a). In the embodiment, the amount of OASL1 protein is measured using ELISA or Western blotting by SDS-PAGE.

Another embodiment provides a method for screening an antiviral agent for combined administration, comprising the steps of: (a) measuring the amount or activity of OASL1 protein after injecting an antiviral agent into cells that are infected with a virus or viral analogue; (b) measuring the amount or activity of OASL1 protein after injecting the antiviral agent and a sample to be assayed into cells that are infected with a virus or viral analogue; and (c) determining the sample to be assayed as an antiviral agent for combined administration if the amount of OASL1 protein in step (b) is less than the amount or activity of OASL1 protein in step (a). In the embodiment, the virus can be any one of dsDNA virus, ssDNA virus, dsRNA virus, (+) ssRNA virus, (−) ssRNA virus, ssRNA-RT virus, and dsDNA-RT virus; the viral analogue can be poly (I:C) or poly (A:U); and the amount of OASL1 protein is measured using ELISA or Western blotting by SDS-PAGE. Another embodiment provides a diagnostic kit for antiviral immunity, comprising primers that correspond to a nucleotide sequence of the Oasl1 gene. In the embodiment, the Oasl1 gene can be any one of SEQ ID NOs: 1 to 7.

Another embodiment provides a method for provision of information on antiviral immunity, comprising the step of PCR with primers that correspond to a nucleotide sequence of the Oasl1 gene. In the embodiment, the Oasl1 gene can be any one of SEQ ID NOs: 1 to 7.

Another embodiment provides a non-human transformant having a deletion of the Oasl1 gene and an enhanced production of antibodies. In the embodiment, the Oasl1 gene can be any one of SEQ ID NOs: 1 to 7; and the transformant is derived from a mammal, more specifically, a mouse.

The Oasl1 gene is homologous in mouse (Mus musculus), human (Homo sapiens), rat (Rattus norvegicus), dog (Canis lupus familiaris), horse (Equus caballus), cattle (Bos Taurus), and pig (Sus scrofa) (Perelygin, A. A., A. A. Zharkikh, S. V. Scherbik, and M. A.

Brinton. The mammalian 2′-5′ oligoadenylate synthetase gene family: Evidence for concerted evolution of paralogous Oas1 genes in Rodentia and Artiodactyla. Journal of Molecular Evolution, 63, 562-576 (2006)).

PCR is a reaction that amplifies the DNA template and consists of a denaturation step, an annealing step, and a polymerization step, where the procedure is repeated for a few dozen cycles. During the denaturation step, double-stranded DNA is divided into single-stranded DNA; during the annealing step, the primer specifically binds to the (single-stranded) DNA template; and during the polymerization step, the DNA that is complementary to the DNA template is polymerized by the DNA polymerase. The kit used in the present invention comprises dNTP and DNA amplification reaction buffer. The composition of the buffer may vary according to the type of DNA polymerase selected, etc. The kit of the present invention may be provided in a concentrate form or in a form that does not require dilution. Once the DNA sample for detection is added to the kit and PCR is performed, the Oasl1 gene, if present, will be amplified and the presence of Oasl1 can be confirmed by means of, for example, electrophoresis.

It is desirable that the aforementioned compounds of the present invention, which are used in diagnostic compositions, are labeled to be detectable. A variety of techniques for labeling biomolecules are well known to a person skilled in the art and are considered to be within the scope of the present invention. Such techniques are described in: Tijssen, P. “Practice and Theory of Enzyme Immunoassays.” Laboratory Techniques in Biochemistry and Molecular Biology. Vol. 15. Ed. R. H. Burdon and P. H. van Knippenberg, New York: Elsevier Science Ltd, 1985; Davis L. G., M. D. Dibmer, and J. F. Battey, eds. Basic Methods in Molecular Biology. Elsevier, 1986; Mayer, R. J. and J. H. Walker, eds. Immunochemical Methods in Cell and Molecular Biology. London: Academic Press, 1987; or in the series, Methods in Enzymology. Academic Press, Inc.

There are many different methods of labeling besides those known to a person skilled in the art. Examples of labeling methods that can be used in the present invention are enzymes, radioactive isotopes, colloidal metals, fluorescent compounds, chemiluminescent compounds, and bioluminescent compounds.

Commonly used labels include fluorescent substances (e.g., fluorescein, rhodamine, Texas Red, etc.), enzymes (e.g., horse radish peroxidase, (β-galactosidase, and alkaline phosphatase), radioactive isotopes (e.g., ³²P and ¹²⁵I), biotin, digoxygenin, colloidal metals, and chemiluminescent or bioluminescent compounds (e.g., dioxetanes, luminols, and acridiniums). Labeling procedures, such as covalent coupling of enzymes or biotinyl groups, iodinations, phosphorylations, biotinylations, and the like, are well known in the art.

Detection methods include, but are not limited to, autoradiography, fluorescence microscopy, direct and indirect enzymatic reactions, etc. Commonly used detection assays can include radioisotopic or non-radioisotopic methods. These include, inter alia, Western blotting, overlay assay, Radioisotopic Assay (RIA) and Immune Radioimmunometric Assay (IRMA), Enzyme Immuno Assay (EIA), Enzyme Linked Immuno Sorbent Assay (ELISA), Fluorescent Immuno Assay (FIA), and Chemioluminescent Immune Assay (CLIA).

Besides the aforementioned active ingredients, the preparation can additionally comprise one or more types of pharmaceutically acceptable carriers for administration. Pharmaceutically acceptable carriers include saline, sterile water, Ringer's solutions, buffered saline, dextrose solutions, maltodextrin solutions, glycerol, ethanol, and a mixture of one or more of these ingredients. In addition, by further adding antioxidants, buffers, bacteriostats, and lubricants, preparations can be made for injectable formulations, such as aqueous solutions, suspensions, and emulsions, or for pellets, capsules, granules, or tablets, as necessary. Furthermore, suitable preparation methods in the art for each disease or ingredient can be achieved by using methods described in Remington's Pharmaceutical Sciences (latest edition), Easton, Pa.: Mack Publishing Company.

The compositions of the present invention may be administered to a human or animal via a variety of routes including parenteral, intraarterial, intradermal, transcutaneous, intramuscular, intraperitoneal, intravenous, subcutaneous, oral, and intranasal routes of administration. The dosage may vary according to the patient's weight, age, sex, general health, diet, time and mode of administration, excretion rate, and severity of disease. Daily dosage of the composition is about 10 ng/kg to 10 mg/kg, preferably about 80 ng/kg to 400 ng/kg, once a day or more preferably spread out over multiple times a day.

### Advantageous Effects

By the above means, the present invention can screen for antiviral agents that can reduce the amount of expression of OASL1 protein, and furthermore, enhance immunity against viruses by suppressing the expression of the OASL1 protein.

### BRIEF DESCRIPTION OF THE DRAWINGS

FIGS. 1A-D concern Oasl1-knockout mice according to an embodiment of the present invention. a) Genetic map before and after genetic modification. b) Southern blot analysis of genomic DNA extracted from mouse tails and digested with EcoRI. c) RT-PCR analysis of OASL1 mRNA in wild-type and Oasl1^{-/-} bone marrow-derived macrophages (BMDMs). d) Western blot analysis of OASL1 protein in wild-type and Oasl1^{-/-} BMDMs.

FIGS. 2A-D illustrate the expression of type I interferon according to an embodiment of the present invention. a,b,c) Control (0 h) or treated with poly (I:C). a,b) qPCR analysis of extracted RNA. c) ELISA analysis of IFNα/β and TNFα. d) Whole-genome microarray analysis of RNA extracted from BMDMs at 9 h after treatment with poly (I:C).

FIG. 3 presents graphs showing qPCR analysis of the amount of RNA in BMDMs left untreated or treated for 9 h with poly (I:C) according to an embodiment of the present invention.

FIGS. 4A-B illustrate qPCR analysis of the amount of RNA (a), or cytometric bead array analysis of the amount of cytokines (b), in BMDMs treated with poly (I:C) according to an embodiment of the present invention.

FIGS. 5A-B present graphs showing qPCR analysis of the amount of RNA in BMDMs left untreated or treated with EMCV (a) or HSV-1 (b) according to an embodiment of the present invention.

FIGS. 6A-C illustrate the expression of IRF3 and IRF7 mRNAs and proteins according to an embodiment of the present invention. a,b,c) Control (0 h) or treated for 9 h with poly (I:C). a) qPCR analysis of the amount of IRF3 and IRF7 mRNAs. b) Western blot analysis of IRF3 and IRF7 proteins. c) Western blot analysis of IRF3 and IRF7 proteins after classifying them into the nucleus, cytosol, and whole cell.

FIG. 7 presents graphs showing immunoblot analysis of half-life of IRF7 protein according to an embodiment of the present invention.

FIG. 8 presents graphs showing the inhibition of translation of IRF7 mRNA by OASL1 in BMDMs treated with poly (I:C) according to an embodiment of the present invention. Top left: Immunoblot analysis of equal volumes of samples obtained from polysomal fractions 4-16 (C is positive control). Other: Quantitative qPCR analysis of IRF3, IRF7, and TNFα for each fraction.

FIG. 9 presents graphs showing qPCR analysis of each gene's mRNA in 16 polysomal fractions obtained from BMDMs treated for 12 h with poly (I:C) according to an embodiment of the present invention.

FIGS. 10A-B illustrate that the inhibition of translation of IRF7 mRNA by OASL1 according to an embodiment of the present invention is a general phenomenon. a,b) Top panel: Western blot analysis. Bottom panel: qPCR analysis.

FIG. 11 presents graphs showing immunoblot analysis (top panel) and qPCR analysis (bottom panel) of the expression of IRF7, IRF3, and HDAC1 proteins and mRNAs in WT and Oasl1-KO BMpDCs left untreated or treated for 12 h with CpG-A (3 μM) or R848 (2 μg/ml) according to an embodiment of the present invention.

FIG. 12 illustrates immunoblot analysis (top panel) and qPCR analysis (bottom panel) of the expression of IRF7 protein and mRNA in WT and Oasl1-KO mice treated for 9 h with PBS, poly (I:C) (100 μg/mouse), or LPS (100 μg/mouse) according to an embodiment of the present invention.

FIGS. 13A-C presents graphs showing an increased production of type I interferon, as well as increased resistance to the virus, in Oasl1^{-/-} mice after treatment with poly (I:C) (a), infection with EMCV (b), or infection with HSV-1 (c), according to an embodiment of the present invention.

FIG. 14 presents graphs showing cytometric bead array analysis of IL6, IL10, MCP1, and IFNγ proteins measured every hour in Oasl1^{-/-} mice after treatment with poly (I:C) (100 μg/mouse) according to an embodiment of the present invention.

FIG. 15 presents graphs showing the heart viral titer measured in WT and Oasl1^{-/-} mice 4 days after infection with EMCV (100 PFUmouse) according to an embodiment of the present invention.

### BEST MODE FOR CARRYING OUT THE INVENTION

Hereinafter, the elements and technical features of the present invention are described in detail through the following examples. However, these are only intended to illustrate the present invention and do not limit the scope of the invention.

### Modes for Carrying Out the Invention

### Example 1

### Production of Type I Interferon

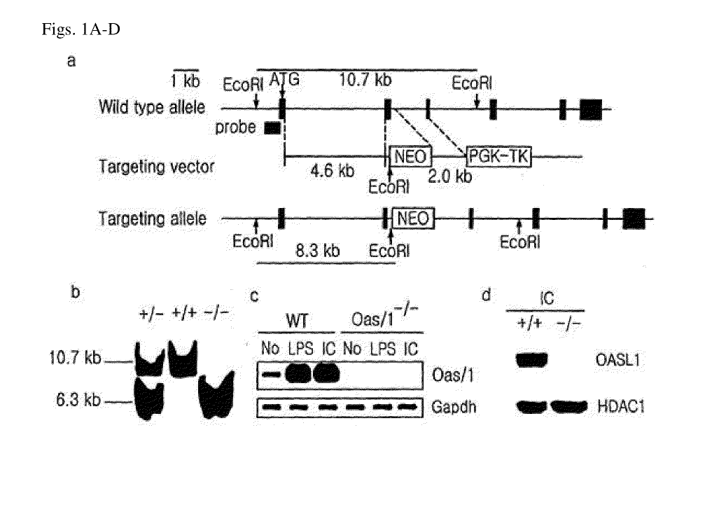

To investigate the physiological role of OASL1 protein, Oasl1-knockout mice were generated using the standard gene-targeting strategy with embryonic stem cells (FIG. 1). Mice of 6 to 10 weeks of age were used that were crossed more than five times onto the C57BL/6J background. All mice derived from the two independent embryonic stem cell clones had identical phenotypes and were grown pathogen-free.

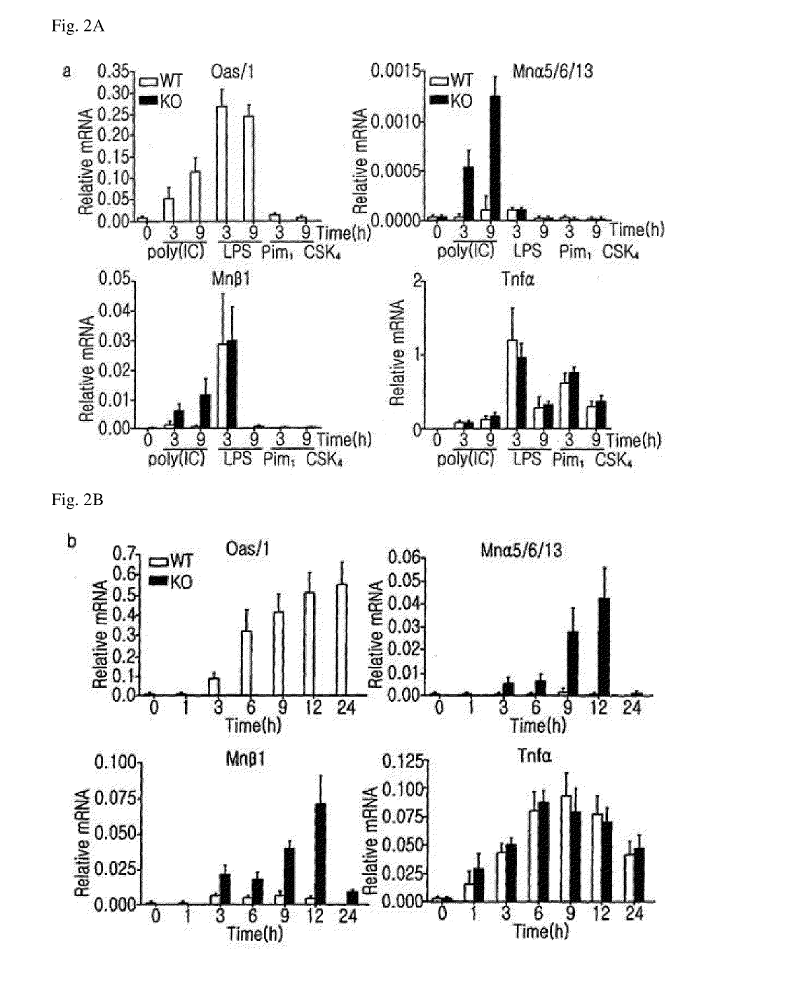

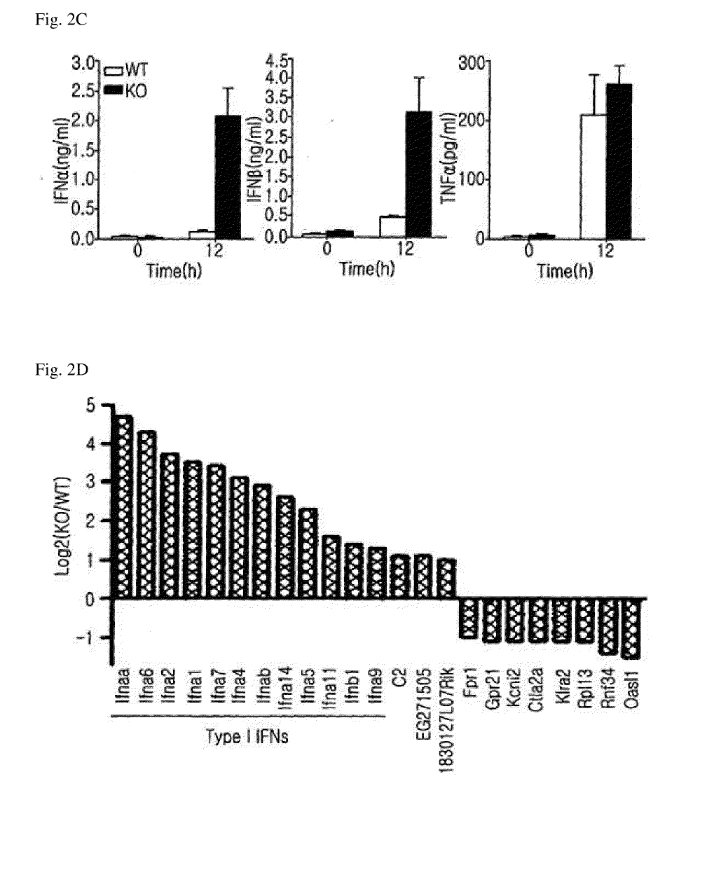

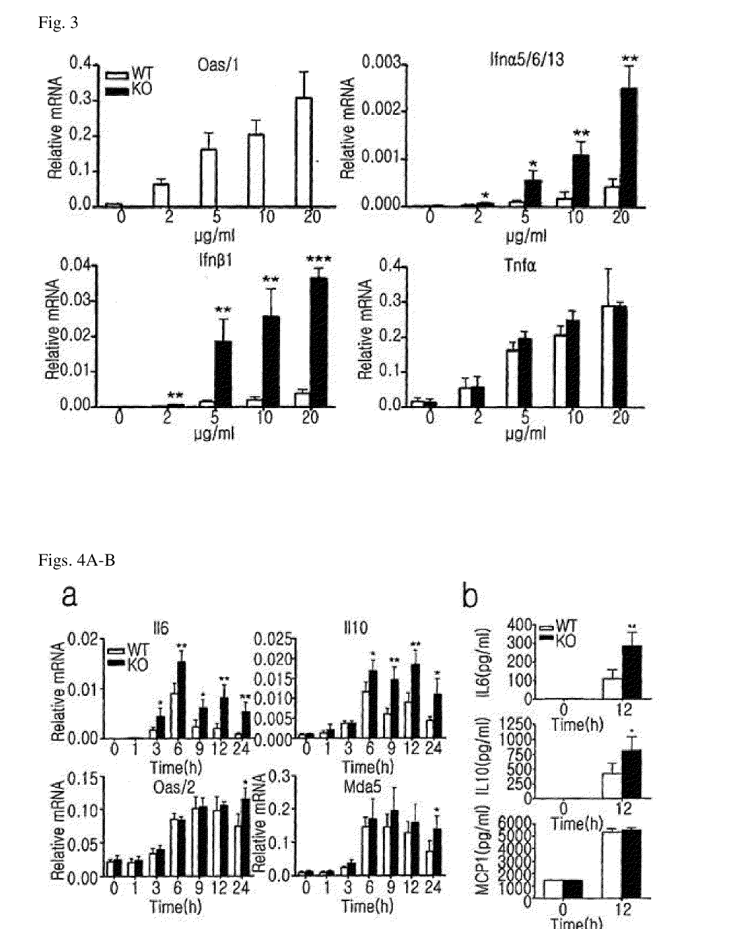

Oasl1 can be induced in BMDM by interferon-inducing pathogen-associated molecular patterns (PAMPs), such as LPS and poly (I:C). Therefore, the expression of type I interferons, such as IFNα5/6/13 and IFNIβ1, was measured after treatment with these PAMPs. The results showed that when treated with poly (I:C), Oasl1^{-/-} BMDMs had a much higher expression of type I interferons than did wild-type BMDMs (FIG. 2). Similar results were obtained from a broad range of doses (2 μg/ml to 100 μg/ml) of poly (I:C) (FIG. 3). Furthermore, as doses of poly (I:C) increased, the expression of IFNα mRNA also increased.

To examine a critical time point to regulate type I interferons and the specificity of such regulation, the inventors measured the induction process for the expression of type I interferons, IFN-stimulated genes (ISGs), and a few major inflammatory cytokines, using real-time PCR (qPCR).

The high expression of type I interferon in Oasl1^{-/-} BMDMs treated with Poly (I:C) peaked in the late phase (9-12 h) where a type I interferon-mediated positive feedback loop robustly induced the expression of ISGs and type I interferon genes (FIG. 2b). However, there was no substantial difference in inflammatory cytokine TNFα between wild-type and Oasl1^{-/-} BMDMs; some difference in IL6 and IL10 was observed in the late phase (FIGS. 2b and 4). With regard to ISGs, including OASL2 and MDA5, a significant difference between wild-type and Oasl1^{-/-} BMDMs was only observed in the end phase (FIG. 4); therefore, the difference was considered to have been indirectly caused by the large amount of type I interferons produced in the early phase in Oasl1^{-/-} BMDMs. The same aspect was also found in protein expressions measured in culture supernatants (FIG. 2c). Thus, the results indicated that the strongly induced OASL1 somewhat specifically inhibited the expression of type I interferons.

To determine whether the high expression of type I interferons in Oasl1^{-/-} cells was a specific phenomenon, the expression patterns of various genes were measured in wild-type and Oasl1^{-/-} BMDMs at 9 h after treatment with a low dose (5 μg/ml) of poly (I:C). Only 23 transcripts out of approximately 35,000 transcripts had a signal difference of greater than twofold in wild-type versus Oasl1^{-/-} BMDMs; 15 had higher signals in Oasl1^{-/-} cells, whereas 8 had lower signals in Oasl1^{-/-} cells (FIG. 2d). Most genes (12 out of 15 up-regulated genes) with greater than twofold higher expressions in Oasl1^{-/-} BMDMs encoded type I interferons; genes with greater than 4-fold higher expressions in Oasl1^{-/-} BMDMs encoded only type I interferons (9 genes). Thus, the results indicated that type I interferon was the main gene whose mRNA was affected when treating Oasl1-deficient BMDMs with poly (I:C).

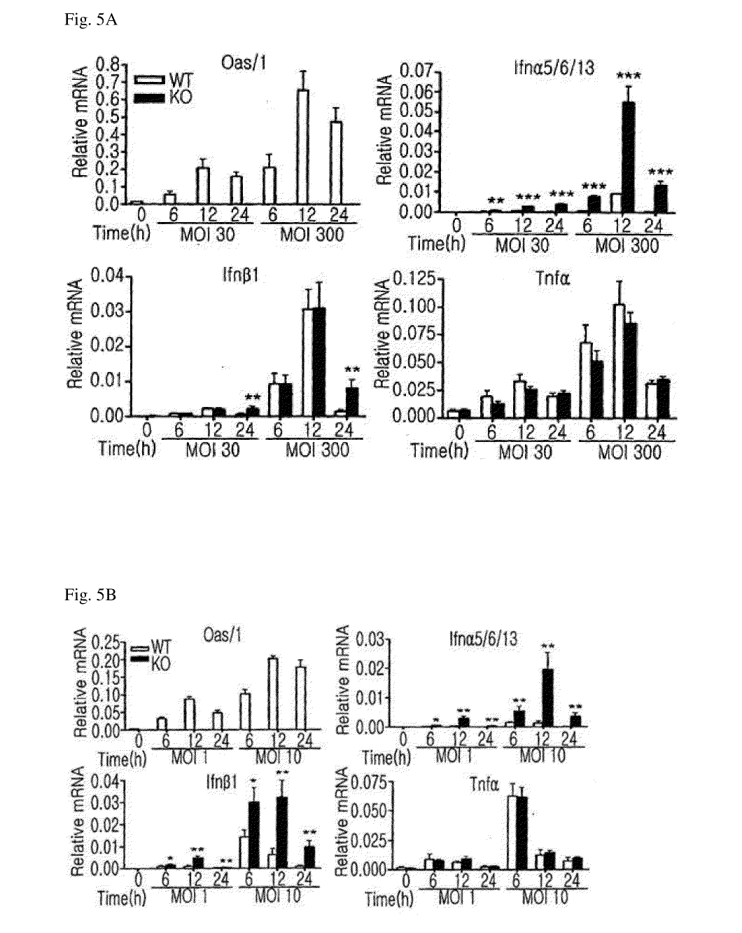

In addition, to determine whether actual viral infection would produce a similar result, BMDMs were treated with encephalomyocarditis virus (EMCV), an RNA virus recognized by MDA5, or with herpes simplex virus 1 (HSV-1), a DNA virus recognized by IFI16. The results showed that the expression of type I interferon mRNA was more than 5-fold higher in Oasl1^{-/-} BMDMs than in wild-type BMDMs, while the expression of TNFα mRNA did not differ, as illustrated in FIG. 5. Although the difference in IFNβ1 was much smaller after infection with EMCV, the trend was similar to that which could be observed in poly (I:C) treatment. Thus, the results indicated that OASL1 efficiently inhibited type I interferons, especially IFNα, during viral infection.

### Example 2

### IRF7 and IRF3 Protein and mRNA Expressions

As described in the Example 1, the genes encoding type I interferons were affected the most in Oas -knockout BMDMs treated with poly (I:C), where the transcription factors (TFs) that have the greatest effect on the expression of type I interferon mRNA are IRF3 and IRF7. Therefore, the inventors investigated whether there was a change in the expression of IRF3 and IRF7 mRNAs and proteins in Oasl1^{-/-} BMDMs treated with poly (I:C).

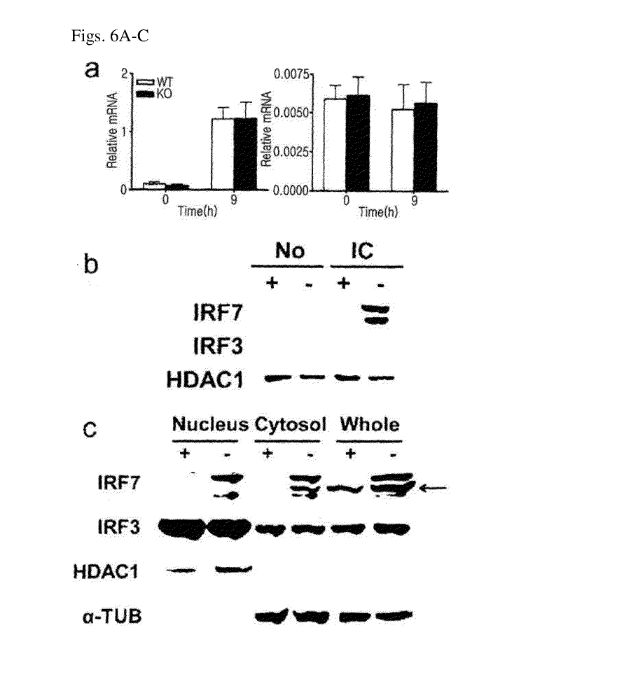

As illustrated in FIG. 6, the results showed that the expression of mRNA did not differ significantly in wild-type versus Oasl1^{-/-} BMDMs at 9 h after treatment with poly (I:C) (FIG. 6a). However, Oasl1^{-/-} BMDMs had an approximately 6.5-fold greater amount of IRF7 protein than did wild-type BMDMs, while the amount of IRF3 protein was similar in both cells (FIG. 6b).

Furthermore, in order to measure the activation level of these proteins, the inventors measured the degree of translocation of IRF3 and IRF7 into the nucleus. There was no significant difference between the two types of cells in the translocation of IRF3 protein. However, with regard to IRF7 protein, Oasl1^{-/-} BMDMs showed an approximately 6.5-fold higher level than did wild-type BMDMs (FIG. 6c). Thus, the results indicated that the activation process of IRF7 was irrelevant to the presence of deletion of Oasl1^{-/-}; in addition, the change that was induced by the deletion of Oasl1 in BMDMs treated with poly (I:C) was the increased expression of IRF7.

### Example 3

### Mechanism of the Inhibition of IRF7 Protein

The results from Example 2 could be explained by the following two possibilities: either IRF7 mRNA was more efficiently translated in Oasl1^{-/-} BMDMs treated with poly (I:C) than in wild-type BMDMs, or IRF7 protein was more stable in Oasl1^{-/-} BMDMs.

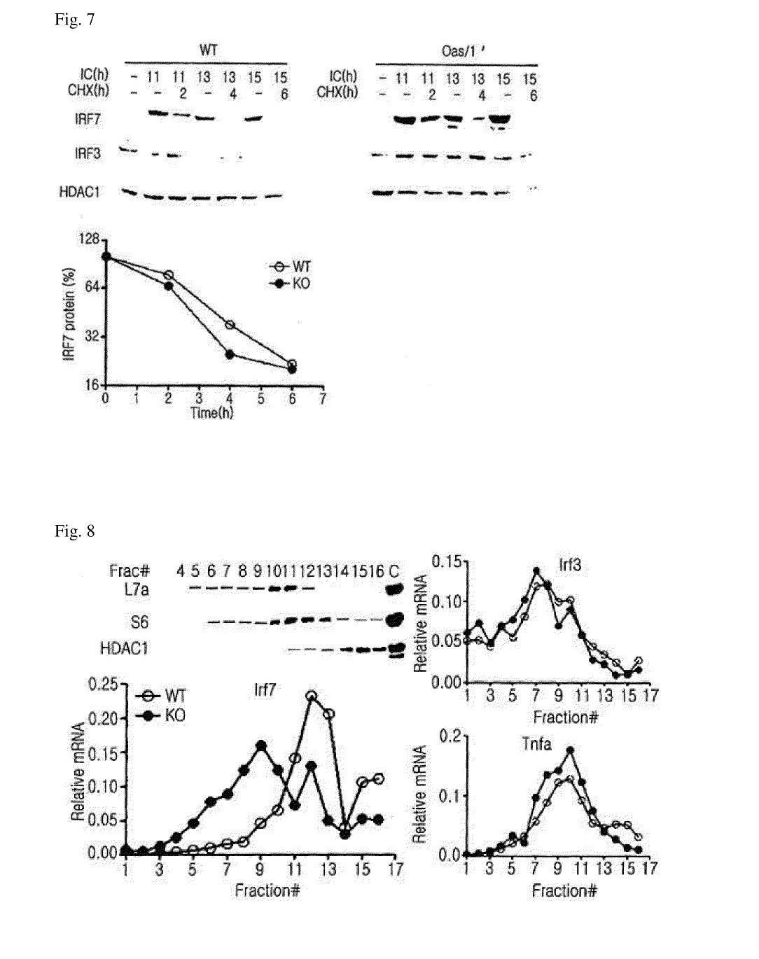

To determine whether the IRF7 protein was more stable in Oasl1^{-/-} BMDMs, the half-life of IRF7 protein was measured after using cycloheximide (CHX) to inhibit the translation into protein. As illustrated in FIG. 7, the half-life of IRF7 protein in wild-type and Oasl1^{-/-} BMDMs was 3 h and 2.5 h, respectively, thus showing similarity. The results indicated that there was no difference in the stability of IRF7 protein.

To determine whether IRF7 mRNA was more efficiently translated in Oasl1^{-/-} BMDMs, the amount of IRF7 mRNA associated with polysomes, having robust translations, was compared. As illustrated in FIG. 8, more than 50% of IRF7 mRNA was found in polysomal fractions (fractions 1-9) in Oasl1^{-/-} BMDMs, whereas about 90% of IRF7 mRNA in wild-type BMDMs was found in monosomal, subribosomal, or soluble fractions (fractions 10-16). Furthermore, as illustrated in FIGS. 8 and 9, the two types of cells showed no significant difference with respect to mRNAs other than IRF7 mRNA, such as those of IRF3, TNFα, IFNβ1, OASL2, IL6, and IL10. The results suggested that OASL1 specifically inhibited the translation of IRF7 mRNA.

### Example 4

### Generality of the Control of IRF7 Translation by OASL1

A test was conducted whether the inhibition of translation of IRF7 mRNA by OASL1 was specific to poly (I:C) treatment or general in BMDMs. Because BMDMs contain a variety of nucleic acid sensors in the intracellular space besides TLR3 and TLR4, the total amount of IRF7 protein and mRNA was measured using Western blot and qPCR after extracellular treatment with IFNβ, poly (I:C), or LPS, and intracellular treatment with nucleic acids (poly (I:C), poly(dA)·poly(dT), and plasmid DNA).

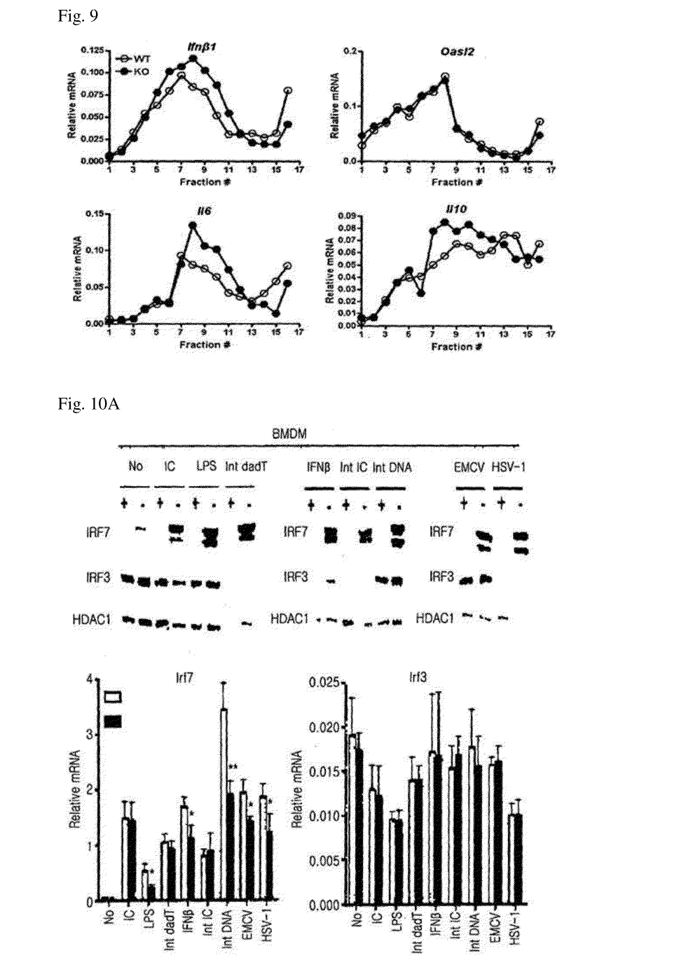

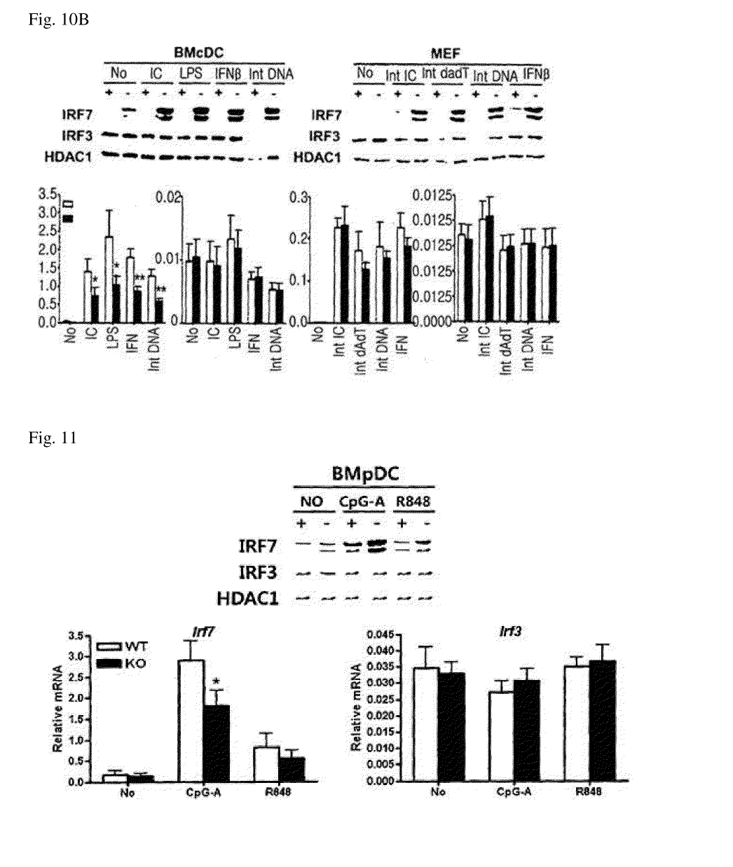

As illustrated in FIG. 10a, the amount of IRF7 protein, after treatment with interferon-inducing PAMPs and interferons that increase the expression of OASL1 and IRF7 mRNAs in BMDMs, was more than 5-fold greater in Oasl1^{-/-}BMDMs than in wild-type BMDMs at 12 h after treatment. Thus, the results were similar to those of the above example involving poly (I:C). However, the amount of IRF7 mRNA was not greater in Oasl1^{-/-} BMDMs than in wild-type BMDMs.

In addition, Oasl1^{-/-} BMDMs produced more than 5-fold greater amount of IRF7 protein also during infection with EMCV and HSV-1.

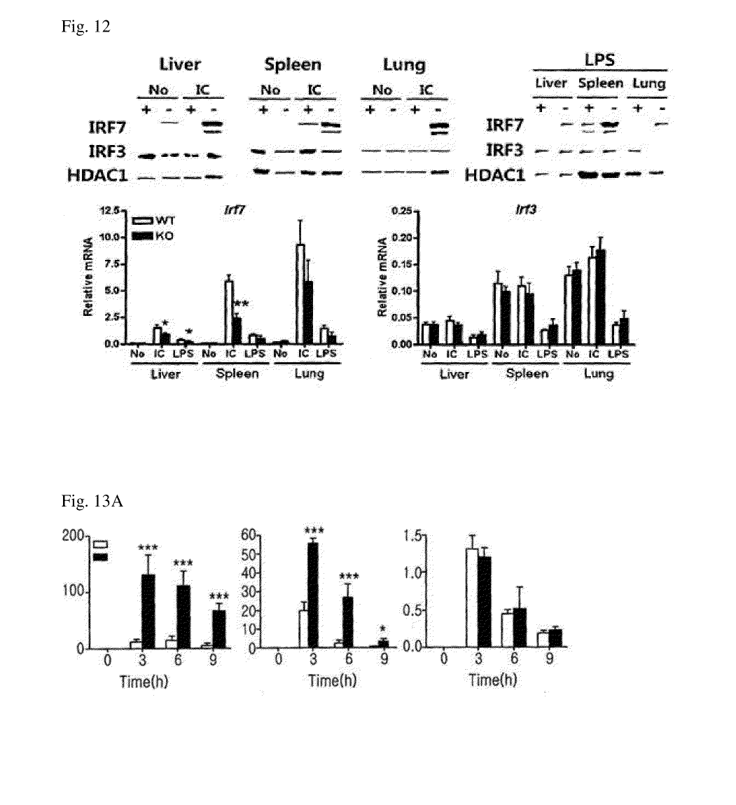

A test was conducted whether the inhibition of translation by OASL1 could also be observed in other major innate immune cells (BM conventional DCs (BMcDCs) and BM plasmacytoid DCs (BMpDCs)) and non-immune cells (mouse embryonic fibroblasts (MEFs)). As illustrated in FIG. 10b, the expression of IRF7 protein was more than 5-fold greater in Oasl1^{-/-} BMDMs than in wild-type BMDMs because all BMcDCs, which express TLR3, TLR4, and at least IFI16 (non-AT-rich DNA sensor) among intracellular nucleic acid sensors, and all MEFs, which express intracellular nucleic acid sensors but not TLRs, responded to the stimulation by all ligands of the same kind. However, there was no significant difference in the amount of IRF7 mRNA. A similar result (a greater than 3-fold increase in the KO cells) was obtained with BMpDCs (FIG. 11). Moreover, Oasl1^{-/-} BMDMs had a greater than 3-fold increase in the expression of IRF7 protein in other tissues including the liver, spleen, and lung (FIG. 12).

### Example 5

### Expression of Type I Interferon in Oasl1^{-/-} Mice (In Vivo)

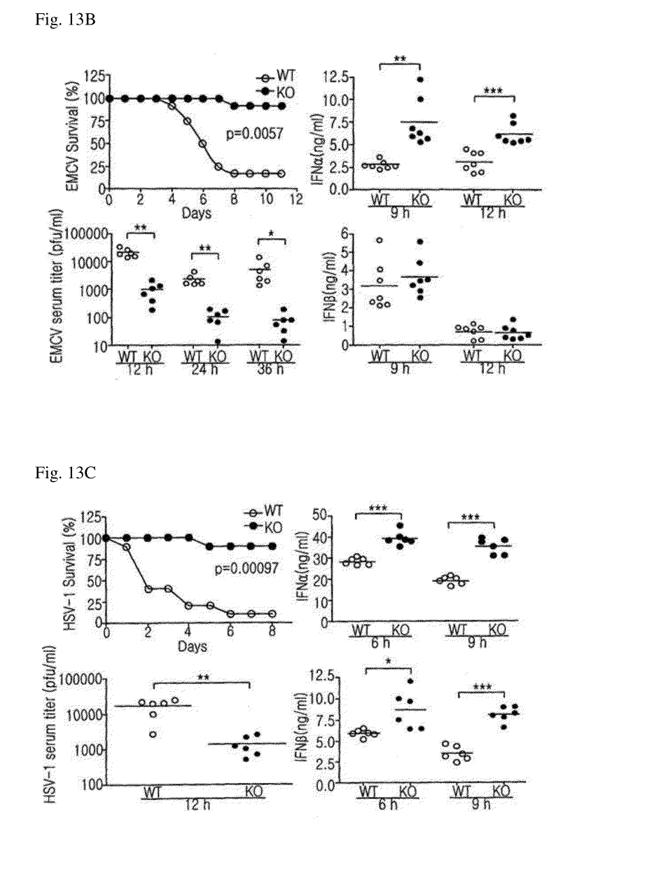

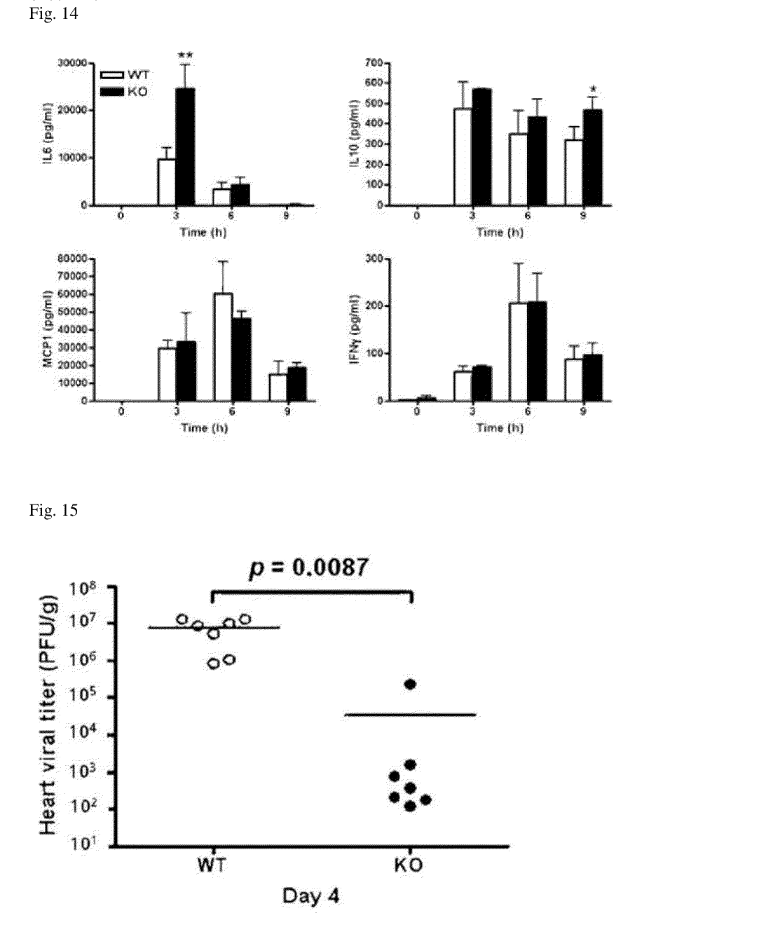

A test was conducted whether the expression of type I interferons increased in vivo after treatment with poly (I:C), as observed in Oasl1^{-/-} BMDMs. As illustrated in FIG. 13a, Oasl1^{-/-} mice produced a greater amount of type I interferons, especially IFNα, when treated with poly (I:C). In addition, the amount of IL6 protein produced was slightly greater in Oasl1^{-/-}BMDMs but there was no difference in the production of TNFα protein, as illustrated in FIG. 14.

As illustrated in FIG. 13b, Oasl1^{-/-}mice had a higher survival rate relative to that of wild-type mice when infected with EMCV, as well as an increased production of type I interferons, especially IFNα, and a lower serum viral titer. In addition, as illustrated in FIG. 15, the heart viral titer in the late phase of infection was considerably lower in Oasl1^{-/-} mice.

Thus, the results indicated that during infection with EMCV, Oasl1^{-/-} mice produced a greater amount of type I interferons in the early phase of infection (within 12 h after infection), where type I interferons, by suppressing virus replication, allowed Oasl1^{-/-} mice to clear the viruses more efficiently and to achieve better survival from the deadly infection.

To determine whether the enhanced defense capacity demonstrated by Oasl1^{-/-} mice was limited to EMCV infection only, Oasl1^{-/-} mice were infected with HSV-1, a DNA virus of a different form. As in the case of EMCV infection, Oasl1^{-/-} mice showed a higher survival rate, produced a greater amount of type I interferons, and had a lower serum viral titer than did wild-type mice when infected with HSV-1 (FIG. 13c). Thus, the results indicated that Oasl1^{-/-} mice could achieve enhanced resistance to the viruses by overproducing type I interferons relative to wild-type mice. The results also suggested that Oasl1^{-/-} mice would demonstrate enhanced resistance to most viral infections as a result of the increased activation of IRF7.

What is claimed is:

1. An antiviral pharmaceutical composition comprising, as active ingredients, antisense or siRNA oligonucleotide that has a sequence complementary to a nucleotide sequence of the Oasl1 gene.

2. The antiviral pharmaceutical composition according to claim 1, wherein the nucleotide sequence of the Oasl1 gene has any one of SEQ ID NOs: 1 to 7.

3. A method for screening an antiviral agent, comprising the steps of:
(a) measuring the amount or activity of OASL1 protein in cells;
(b) injecting into cells a sample to be assayed;
(c) measuring the amount or activity of OASL1 protein in cells of step (b); and
(d) determining the sample to be assayed as an antiviral agent if the amount or activity of OASL1 protein in step (c) is less than the amount or activity of OASL1 protein in step (a).

4. The method for screening an antiviral agent according to claim 3, wherein the amount of OASL1 protein is measured using ELISA or Western blotting by SDS-PAGE.

5. A method for screening an antiviral agent for combined administration, comprising the steps of:
(a) measuring the amount or activity of OASL1 protein after injecting an antiviral agent into cells that are infected with a virus or viral analogue;
(b) measuring the amount or activity of OASL1 protein after injecting the antiviral agent and a sample to be assayed into cells that are infected with a virus or viral analogue; and
(c) determining the sample to be assayed as an antiviral agent for combined administration if the amount of OASL1 protein in step (b) is less than the amount or activity of OASL1 protein in step (a).

6. The method for screening an antiviral agent for combined administration according to claim 5, wherein the virus is any one of dsDNA virus, ssDNA virus, dsRNA virus, (+) ssRNA virus, (−) ssRNA virus, ssRNA-RT virus, and dsDNA-RT virus.

7. The method for screening an antiviral agent for combined administration according to claim 5, wherein the viral analogue is poly (I:C) or poly (A:U).

8. The method for screening an antiviral agent for combined administration according to claim 5, wherein the amount of OASL1 protein is measured using ELISA or Western blotting by SDS-PAGE.

9. A diagnostic kit for antiviral immunity, comprising primers that correspond to a nucleotide sequence of the Oasl1 gene.

10. The diagnostic kit for antiviral immunity according to claim 9, wherein the Oasl1 gene is any one of SEQ ID NOs: 1 to 7.

11. A method for provision of information on antiviral immunity, comprising the step of PCR with primers that correspond to a nucleotide sequence of the Oasl1 gene.

12. The method for provision of information on antiviral immunity according to claim 11, wherein the Oasl1 gene is has any one of SEQ ID NOs: 1 to 7.

13. A non-human transformant having a deletion of the Oasl1 gene and an enhanced production of antibodies.

14. The transformant according to claim 13, wherein the Oasl1 gene is has any one of SEQ ID NOs: 1 to 7.

15. The transformant according to claim 13, wherein the transformant is derived from a mammal.

16. The transformant according to claim 13, wherein the transformant is derived from a mouse.


SEQUENCE LISTING (7 sequences)

SEQ ID NO: 1 - DNA, 2124 bp, Mus musculus
agagccatgctcccaagcttctctcttcacccccctttccgccattgggtgccagaggtcagctggaaaggagccgccttaaaaaaggaggagccatggcagtcgcccaggagctgtacggcttcccggcctccaagctggactcctttgtggctcagtggctgcagccaaccagagagtggaaagaagaggtcctggagaccgtgcagacagtggagcagttcctgaggcaggagaatttccgtgaagatcgtggcccggctcgggatgtgcgcgtgctcaaggtactcaaggtaggctgctttgggaatggcaccgtgctcaggagcactacagacgtggagctggtcgtgttcctgagctgtttccacagcttccaggaagaagccaagcaccatcaggctgtcctgagactgatacagaaaaggatgtactactgccaggagctgatggaccttgggctcagtaacctgagtgtgactaacagagtacccagtagtctcatcttcacgatccagaccagggagacctgggagaccatcactgtcaccgttgtgcccgcctacagagccctgggcccttcctgtcccagctccgaggtctacgcaaatctgatcaaggctaatgggtacccaggaaatttctctccatccttcagcgagctgcagcgaaacttcgtgaagcatcggccgacgaagctgaagagcctccttcggttggtcaaacactggtaccagcagtatgtgagagacaagtgcccccgggccaacctgccccctctctatgccctggagctgctcactgtctatgcctgggaagcgggcacccgggaggatgccaacttcaggctggatgaaggcctcgccacggtgatggagctgctccaggatcatgagctcctctgtatctactggaccaagcactacacgctgcagcacccggtcatcgaggcctgtgtcaggagacagctcaggggacaaaggcctatcatcctggacccagcagaccccaccaacaatgtggcagaaggctacagatgggacatagtggctcagcgggccaaccagtgtctgaaacaggactgttgctatgacaacagggacagccccgtccccagctggagggtgaagagagcacccgatatccaggtgaccgtgcaggagtgggggcactcggatttaaccttctgggtgaacccttatgaacccataaagaaactgaaagagaaaatccaactgagccagggctacttgggcctgcagcgtctgtcctttcaggagcccggcggagaacgtcagctcatcagaagccattgcacgctcgcctactacggaatcttctgcgacacccacatctgcctgctggacaccatctcccctgagatccaggtctttgtgaaaaacccggatggcaggagccacgcctatgcgatccacccgcttgattacgtcctgaacctgaagcagcagatagaagacaggcagggccttcgatgccaggagcagcgcctggagttccagggccacatcctggaggactggtttgactttaaatcctatggcatccaagacagtgtcacagtcatcctgtccaagacgacggagggggcagctccatttgtgcccagctagcttcctctgtcggtggctctgcctgttttattgtctcatcctagactcagcctagttgcctctccttcccgtcctctgcccggatggtccacgtcttcagtaccttgccagcagggagtcagagggggtgtgagaagtcgtgtacagccagacactcttgtgtgacaatggaattctgcagtcccctgggaagtcatgccaggacctctgccttcctcgtggcctcactgtcaagactgtgtcagtgaatagctggcctcacagactattctcacatgttcagagaaagccaaaccatcttcctaaccaatcacagggaccctgcttgaggttgtcccacctccaaattcttcccagtgacctccatcagggcggctctgaagccttcccctgtgcccccaaccacttctgcctgccttcgactatccaaggcaaggtaggaggggatcaagttcctttcaaatggagaataaaaaagccattgtttcttccc

SEQ ID NO: 2 - DNA, 1826 bp, Homo sapiens
acagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcagtatgtgaaagccaggtcccccagagccaatctgccccctctctatgctcttgaacttctaaccatctatgcctgggaaatgggtactgaagaagacgagaatttcatgttggacgaaggcttcaccactgtgatggacctgctcctggagtatgaagtcatctgtatctactggaccaagtactacacactccacaatgcaatcattgaggattgtgtcagaaaacagctcaaaaaagagaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaa

SEQ ID NO: 3 - DNA, 1980 bp, Rattus norvegicus
gcgcagacatggcagtagcccaggagctttacagcttcccagcctccaagctggactcctttgtggctcagtggctgcagccaaccagagaatggaaggaggaggtcctggagacggtgcagacagtggagcagttcctgaggcaggagaacttccgtggagaacgtggcccggcccaggatgtacgagtgctcaaggtactcaaggtaggctgctttgggaatggcacagtactcaggagtaccacagacgtggagctggtggtgttcctgagctgtttccacagcttccaggaagaggccaaacaccaccaggctgttctgagactgatacagaaaaggatgtcttactgccgggacctgctggatctcgggctcagtaacctgagtgtgattgaagaagtgcccagtagtctcatcttcactatccagaccagggagacctgggagcccatcactgtcaccatcgtgcccgccttcagagccctgggaccttcctgtcccaactccgccgaggtctatgtgaatctgatcaaggctaacggctaccccggaaatttctctccttccttcagcgagctacagaggagcttcgtgaagcataggccgactaagctgaagagcctcctacggttggtcaaacactggtaccagcagtacgtgagagacaagtgcccccgggccaacctgccccccctctatgccctggagctgctaactgtctacgcgtgggaagcaggtacgcaggaggattcgaacttcaggctggatgaaggtctcgccactgtcatggagttgctccaggatcatgaactcctgtgcatctactggaccaagtactacaccctgcaacacccagtcattgagcgcttcgtcaggagacagctcaaaggagaaaggcccattatcctggacccagcagaccccacccacaacgtggcgcaaggctacaggtgggatatagttgctcagcgcgccagccagtgtctgaaacaggactgttgctatgacgacagggacgcccccgtccccagctggactgtgaagagagcaccatatatccaggtgaccgtgcagcagtggggtcacccggatttaatcctctgggtgaacccttatgaacccataaagaagctgaaagagaaaatccgactgagccggggctactccggcctgcagcgcctgtcctttcaggagcccggcggccaacggcagctcatcagaagccaatgctcgcttgcctactacggaatcttctgcgacactcagatctgcctgctggacaccatctcccccgagatccaggtctttgtgaaaaacccggatggtggaagccacgcctacgccatccacccacttgacttcgtcctgagcctgaagcagcagatcgaagataggcagggccttcaaagccaggagcagcagctggagttccagggccgcgtcctggaagactggtttgactttaaatcctatggcatccaagatagtatcacgatcatcctatccaggaagagggaggggaaagccccatctgcgcccagctagcttcctctgcctcttttgctatctcatcctaaagtcagcctagtcacccctccttccggtcctcagccgggatgatcccagcagggagccagaagggaatactgccagacgctcttgtgtgacaatgaaactctgcagtcacctgtgaaatcacaccaggacctctacgctctcaagactgggtcagtgaatggccgtcccacaaataaactattctcgcttgttcttgggaagccaaacgatcttcctaaccaatcaaatggaccctgcttcaggttgttcccccacacacaccagcaacctccatcaggttggatctgaagccttcccctgtgctcccaaccacttctgcctgcctcagcctatccaaggcaaggtagggtatcaagttcctttcaaatggagaataaacaacctttgtttcctcccagat

SEQ ID NO: 4 - DNA, 1676 bp, Canis lupus familiaris
tactctggctcagagatggcacaggccatggagctgtatgacacccctgcctccaagctggactccttcgtggctcagtggctgcagccccaccggagctggaaagaagagatcctggaggccgtgaagactgtgcaacaattcctgagggaagagcactttgagggggattatgggccggaccaggaggtgcgggtgctaaaagtggtcaaggtaggctcctttgggaatggcacagttctcaggaacactttggaggtggaactggtggtgttcctgagctgtttccacagcttccagcaagaggccgagcaccaccaagccatcctgagtctgatacaaaaaaagctatggtgttgccgtgatctgctggcgcttgggcttgaggacgtggagatcatccagggagtccccgacgctcttgtcttcaccatccagaccaggaggaccgcagagatcatcactgtcaccatcgtgcctgcctacagggccctggggccttctgcttccaactctcagccatatcctgaggtctatgagagtctgattgaggcccaaggtttccctggaaatttctccccatccttcagcgagctgcagagaaacttcgtgaaacatcggccaaccaagctgaagagcctcctacggctagtaaaacactggtatctgcagtatgtgaaagccaagtgccccagggctgcgctgccccctgactatgccctggagctgctgactatctacgcctgggagatgggcactcaagaggatgagagtttcaggctggatgaaggctttaccactgtgatggaactgttccaagagtatgagttcctctgcatctactggaccaagtattacacattccagaacccagtcattgaggattttgtcaggaaacagctcaaaagagacaggcccatcatcctggatccagctgaccccacccacaatgtggcagaagggtacagatgggacatagttgctcagagggcccgccagtgcctgaaacaggactgttgctatgacaacaaggagaaaccagtccccagctggaatgtgaagaaggcacgagacatccaagtgacagtggaacaatggggttattcagatttgatccttagggtgaacccttataagcccataaagaagattcaagagaagatgtggcaaagccggtgctgctcaggcttgcagcatctgtacctccaggagctgggtgccaagcagcagctcctcagcagccagtactccttggccgattacggtgtcttctccaacactcgcatctgtctggtggagaccaactcccacgagatccaggtctttgtaaagaatcctgatggtgggagcgatgcctacaccactgacgccaaaggcttcatcctgggcctgaaacagcagattgaatacaagcaagggctacccagaaagcagcagcagttggagttccaaggtcaagtcctgaaggattggttgcctttgcagaactatgggatccagcaccgcgacacccttatcctctccaagaagaaagctgaaaggtttccatttctgcccagataatacaggactcagcctatcaaccacttcttccagcccttgcctggggaggtgtggcgtcttcaccagctttggaaacagcctagccctgccagtaggggaacaaggagggatgg

SEQ ID NO: 5 - DNA, 2997 bp, Equus caballus
acagagatggcactgaccccggagctgtatgacactcctgcctccaggctggactcctttgtggcccagtggctacagcccagccgggaatggaaagaagaggtgctggaggcagtgcggaccctggagcagttcctgagggagcagcacttccatgggcagcgtgggctggaccaggaggtgcgggtgctgaaggtggtcaaggtgggctccttcgggaatggcacagtgctcaggaacaccacggaggtggagctggtagtgttcctgagctgtttccacagcttccaggaggaggccgagtaccaccgatccgttctaagcctgatgcggaaaaagctgtggagttgccaggacctgctagacctcaggctcgaggagctgagggtggtccagggagtccccgatgctctcgtcttcaccatccagacctgggggacggcggagcccatcgctgtcactatggtgcctgcctacaaagccctgaggccccctggtcccaactctcggccacaccctgaggtctacgagagcctgattgaggccaacggttaccctggaaatttctccccatccttcagcgagctgcagagaaacttcgtgaagcatcggccaaccaagctgaagagcctcctgcggctggtaaaacactggtacctgcagtatgtgaaagccaggtgccccagggctgcgctgccccctctctatgctctggagctactgactgtctacgcctgggaactgggtactgaagagaatgagaatttcaggttggatgaaggcttcgccactgtgatggagctgctccaggagtatgagttcatctgcatctattggacaaagtactacacgttccagaacccaatcatcgagaactttgtcagaaaacagctcaaaaaagagaggcccatcatcctggatccagctgaccccacccacaacgtggcggaagcgtacagatgggacatagtcgctcagagggcctgccagtgcctgaagcaggactgttgctatgacagggagaacccggtccccagctggaacgtggagagggcacgagacatccaagtgacagtggagcaatggggttacccagacttgattctcacggtgaacccttatgagctcataaggcaggttaaagagaagatccggtggagaagaggctactcgggcgtgcaacgtctgtccttccaggagcccgatggcaagcggcagctcctcagcagccactgctccctggcctattacgggatcttctccaacacttgcatctgtctgctggagaccatctcccccgagatccaggtctttgtgatgaatcctgatggtgggagccacgcttatgccatcgaccccaacagctccatcctgggcctgaagcagcagattgaagacaagcaggggctgcccatgaggcagcaacagctagagtttcgaggccaagtcctgcaggattggctgggtttggggagctatggtgtccaagacagcaacacccttgtcctctccaagaagaaagccagagggactccgtttctacccagttagtttcctctgggaaacctctctgtgcatttctgccatctcatccgggacttattctgtcaaccactccttccagctttctgctgtgaggtcccatgtcttcaccaactttgtaaatggttatcctagccctgatagtggaggactaaggaggggtgtgagagactagacatataaatggacaatggccagaccatatatgacaatagaatactgacccacaatctttgcagccaccagccaggaagccaaaccacaacctgtgcagcaagcaactcaaaacagtgaggacttggtcaataactgggagcttccctaaccaagccccttccaatttaggaccaaccagagaaagccaaacaagctccctaaccaatcacatgggatgctctgcttctaggtagcccacctccagtttctctgcaccaacaacctcacaggcttccctgaagccttctcttttctccaccacaaagcttccccatgaccctgcctgcctctgagcctctgacaaatgcaacggatggtggctcactcccttgctatagcaagtgggaataaataatctttgtttgttctctactgggtggtcttcatgtctttccacagatgctaatgctatccttgtggctgttgcttaatcatttgagcccctctcaactctgggaagtgaggactgtagggaaactgagatgaagataggcaaagtgtgggagagcgagaatgacgcagtgtaaaaacatcccctcaactcctgaaacacacacacaaacagtacctaatggtccaaggactttaggaaaatgacagcatctttaattatcaaagaacatcatattaccaggtttactccaactaaccaataaaaagtttcctttttcccctcccctcttgccctgctttctgatggactacttaccatagaatataaaatgcacttcataattggttctcaacagcctctggacaatttatcaatagtaaccaatttcccacctcttcttgccttcaggggcagaaagagtgcctcctttccgcaggagagacaggacagtccaccctacatcctttatcctttataatgctggatacagccaagttgtaggtggtgggcaaattcttgtttagaaaaaggaataagttgatgtggatcaaggctgccccagggaaagaagcccaaggtgtcctggctgacatgccgatctatatgacccaatacatatctaaagttatacgacgacacaataaccagaccccacctgcactgataccatttaatgactttttacatcatctttccattgtcttgtaaagaaataactcacatacctatgccttataaacttagctttaaccctcaacacattgcagctctttactgcccatgggtcctgtccccatgctactccatgctattttctgaataaaagagcactaccgccagaaaaa

SEQ ID NO: 6 - DNA, 2291 bp, Bos taurus
gtcatcactcagtgtctggggtgcaccagccacagtacacagcctcaataaagaggagccccaggtcagccagacacccttggtttcctgtccactctggtgctttattccacagggtactggagaggagtgttcttaaagaagggtgagtgtgccagcagataggtaggtgcatagctgcatacctagccgccagtggtactctcaagacagatggcagtatccctggagctgtttgataccccagcttctaggctgaactcctttgtggctcagtgccagcataacatcaaggagtggaaagaagacgtggtgaaggctgtgcagtccgtggagaagttcctgaagaagcagcgcttccggggggaccatgggctggaccagaaggtgctgaaggtgatccgggtgggctccttcgggaatggcacggtgctcaggaaccacgcggaggtggagctggtgatgttcctgagcggttttagcagcttccaggaggaagccagcaaccacaatcaccacgagcatgttctgagcatgctctgtgaaaagctcacggatttcccggatctgcttcacctccagccccagaacctgaggctggtccacggagtcacctctgctgtcgcctttaccattcagacctgggagatggaagagcaagtcagtgtcaccattgtgccggcctacggggtcctgaggccttctgttcccaactttcagccctccccagaggtctatgtgagactgattaaggcctgccgtactcttggatacttctccccatccttcagcgagctgcagagaaactttgtgaaatacaagccaaccaagctgaagagcctcctgcggctggtaaaacattggtacctcgagtatgtgaaagccaagtgccctagggcggagctgcccccaatgtatgcccttgagctactgaccatctatgcctgggaaacgggtactgaggagaaagagcgtttcaggttggacaaaggcctggtcactgtgctgctgctgcttacgaagtatcagcgtctctgcatctactggaccaagtactacacattgcagaacccggtcattgaggactttgtcagaaaccagctcaaagaaaagaggcccatcatcttggatctggctgaccccacctacaatgtggcaacaggctacagatgggatatagtggctcagagtgcccgacattgcctgaaacagtcctgttgctatgacaacaatgagacatgcatccccaactggaaactgaagtttgcatgagacatcaacgtgacagtggagcagtggggtcgtgtggatttcgtcatccaggtgaacccctatgagtccatacagaagcttaaagagaagatccagtgtaagctggagtcctcggccccacagcgtttgtccttccaggagcctgggggggagcgacaggtcctcaggaacatctcctttttggcagattacagttttttttttgacgcttgcgtttttttgctgcaaatcgtctttcctgagatgcaggtttttgtgaaaaatcccagcggggggagccacacctttgccatttactccagcagcttattccttgacctgaagtgtcaaattgaagtgagggaggggctactcaaaaagaagcaacagctagagttccaaggccaagtcctgcaggatgagtggagtctgggtttctgtggtgtctgagccagcaccagcctcatcctctccaaaaagacagctgaataggttccatttccacccagttagtctcttctgaaacatctccctgtgcgtttttgctgtttaatctaggtcccatcgatttcacctaaccttgccagtagagggatgacgagggatatgagactaggtatgcaaaccaacaatatccagagcatgtttgacaatagaatttctgacccacaagctctgcagccaacagtctggaagccgaaccattctatcaattaactggaaaccatcaggatttggtcaacaactactagcctccctattccctcccttccratttaggactgatgaaagaaatccaaacaagctccctgaccaatcatataggatgctctgcttctaattagcccacctccagcttcccatgcaaacaacctcagtcggggtatacctgaagccttctctttatgccactataaaactgtctcactgtcctccttgccttggtgtctttgccaaaggcgagggatggtggctaactccctggctatagcgagttggaaataaataaacttcgtttgttctctaaaaaaaaaaaaaaa

SEQ ID NO: 7 - DNA, 1340 bp, Sus scrofa
cttacaggttcaactcagttcactgtgcccctgctgcagagaacagcttccaaggagaggtgtcccaggccagccaggcacccctgttttcctctccactttggtcctttattccagaaggtgctggaggtcagcaggggagccctcttcaagaagtgtgcgtgtgccaggagctgggtaggtgtgtgactagatttccatccaaaagcggtgctcttgagacagatggagctattttacaccccagcttccaagctagacaccttcgtggctaaatgcctgcatccccacaaggagtgtaaagaagaagtgctggaggctgtgaggaccgtgaagaagttcctgtggcaacagtgcttccctgggaagaatgtgcaggtgctagaggtgggctccttcgggaacggcacggtgctccgtgacagcacagaggtggagctggtggcatttctgtgctgtttccgcagcttccgggaaaccgaggacctcgataacatgctggaccagctctctaagacactatgctcttgccagggcctgctggcctttgatctgaaggacgtgtggttggtcgaggaagtcttccgtgccatcgccttcaccatctggaccaagaatttggaagggcccatcactttcaccatcatgcctgcctacagggacctggttccccatggtgggccctccgcagaggtctatgtggatctgattgaggcccgcaagccgcctggaaatttctccccatccttcgccaagttacagagaagttttgtgaaacaccggccagccaagctgaagagcctcctgcgactggtaaaacactggtacctgaagtacgtgaaagccaggtgccccattgctaggctgccccctctctatgccctcgagctactgaccatctatgcctgggaagtgggtacaaaggcacatgagcgtttccatttggacagaggcctcgtcacggtgatgtgcctgctccaggagtatcaatctctctgtatctactggaccaactactacaagttccagaacccaatcattgaggactttgtcagagaacagctcaaaaaagaaaggcctatcatcctggatccggctgaccccacctacaatgtggcctatggctacagatgggacatagtcagtcagagggccagacagtgcctgaaacagtgctgttgccataacaacaagagcccagtccccccgtggtatataaagccttcccactacactgcttgcttcagagtctcagccaaaggctgtgactgattccctggcaataacaattcaggaataaataatctttgttcattctcaaaaaaaaaaaaaaaaaaaaa